(19) United States
(12) Patent Application Publication
(10) Pub. No.: US 2004/0028804 A1
(43) Pub. Date: Feb. 12, 2004

(54) Production of polymeric microarrays

(76) Inventors: Daniel G. Anderson, Framingham, MA (US); Robert Langer, Newton, MA (US)

Correspondence Address: Valarie B. Rosen, Choate, Hall & Stewart, 53 State Street, Exchange Place, Boston, MA, 02109, US

(21) Appl. No.: 10/214,723

(22) Filed: Aug. 7, 2002

(51) Int. Cl. B05D 3/00; C12M 1/34

(52) U.S. Cl. 427/2.11; 435/287.2

(57) ABSTRACT

A method of preparing a microarray of polymer elements. The method includes providing a substrate surface, providing a plurality of individual monomers in a liquid phase, depositing said monomers as a plurality of discrete monomer elements on the substrate surface, and exposing the plurality of discrete monomer elements to initiating conditions to form polymer elements. A portion of the discrete monomer elements may include more than one type of monomer.

### FIELD OF THE INVENTION

This invention pertains to the production of polymeric libraries, and more specifically, to the production of polymeric microarrays.

### BACKGROUND OF THE INVENTION

The further development of many modern technologies is controlled by the rate of materials development. As new materials are developed, they must be tested to see if they have the proper chemical, thermal, mechanical, and other properties for the desired application. However, it takes a long time to produce and test new materials sequentially. It is much more efficient to produce an array of materials and test them in parallel.

U.S. Pat. No. 6,004,617, to Schultz et al., discloses a method of producing and screening an array of compounds. The compounds are produced using standard thin film deposition techniques, such as chemical vapor deposition. U.S. Pat. No. 5,776,359 discloses alternate deposition techniques and a method of using the array to identify magnetoresistive cobalt oxide compounds.

U.S. Pat. No. 5,424,186 discloses a method of synthesizing oligonucleotides on a substrate. Nucleotides are provided with a protecting group and deposited on a substrate. After removal of the protecting group at selected areas of the substrate, a second nucleotide is added to the first. The process is repeated on other areas of the substrate and then on the added nucleotides.

Accordingly, it is desirable to have a method of producing arrays of synthetic polymers on a substrate.

### SUMMARY OF THE INVENTION

In one aspect, the invention is a method of preparing a microarray of polymer elements. The method includes providing a substrate surface, providing a plurality of individual monomers in a liquid phase, depositing said monomers as a plurality of discrete monomer elements on the substrate surface, and exposing the plurality of discrete monomer elements to initiating conditions to form polymer elements. A portion of the discrete monomer elements may include more than one type of monomer.

A portion of the monomers may be a liquid at room temperature; liquid monomers may be combined with a solvent. A portion of the monomers may be a solid at room temperature and may be dissolved in a solvent at a concentration of about 3M or less. The polymer elements may be a first portion of the microarray, and the method may further include repeating the steps of depositing and exposing to prepare a second portion of the array.

The substrate may include glass, plastic, metal, ceramic, and combinations of these. The surface chemistry of the substrate may be modified, for example, by introducing crystallograthic texture, oxidizing, sulfidating, patterning, covalently attaching a functional group, or some combination of these. A polymer may be deposited on the substrate surface. The polymer may be cytophobic, a hydrogel, or both. The polymer elements may be bound to the substrate surface by a covalent or non-covalent interactions and may be biocompatible or non-biocompatible.

One or more drugs, growth factors, combinatorial compounds, proteins, polysaccharides, polynucleotides, or lipids may be included in at least a portion of the polymer element. Such a compound may be covalently attached to at least a portion of the polymer element. For example, the compound may be functionalized with a moiety that is incorporated into the polymer element during polymerization. The functionalized compound is deposited on at least one pre-determined discrete monomer element. Alternatively, or in addition, the compound is incorporated into a backbone of the polymer for noncovalently bound to the polymer of the polymer element.

The polymer elements may be spaced at intervals between about 300 μm and about 1200 μm, less than about 300 μm, less than about 1 μm, or less than about 0.1 μm. Cells may be seeded on the polymer elements. Exemplary cells include yeast cells, mammalian cells, bacterial cells, and plant cells.

The monomers may be deposited with a robotic liquid handling device. The liquid handling device may operate via pin fluid deposition, syringe pumped fluid deposition, or piezoelectric fluid deposition. The monomers may be deposited as drops of between 0.1 nL and about 100 nL, for example, between 1 and 10 nL. Alternatively, the monomers may be deposited as drops of less than about 0.1 nL.

The initiating conditions may include exposure to UV light, exposure to an increase in temperature, exposure to an environment containing water vapor, exposure to an environment containing oxygen, or some combination of these. A chemical initiator may be deposited on the discrete monomer element. For example, the chemical initiator may be co-deposited with at least a portion of the monomers or deposited separately from at least a portion of the monomers. Alternatively, the initiator may be co-deposited with a first portion of the monomers and deposited on the discrete element separately from a second portion of the monomers. Exemplary initiators include radical initiators, redox initiators, thermal initiators, and ionic initiators.

In another aspect, the invention is a microarray of polymers including a plurality of discrete polymer elements bound to a surface. The microarray is produced by the steps of providing solutions of monomers of polymer materials in a liquid phase, depositing at least a first portion of said monomers as a plurality of discreet monomer elements on the surface, depositing at least a second portion of the monomers on a portion of the discrete monomer elements, and exposing the plurality of discrete monomer elements to initiating conditions so that polymer elements are formed.

### BRIEF DESCRIPTION OF THE DRAWING

The file of this patent contains at least one drawing executed in color. Copies of this patent with color drawings will be provided by the Office upon request and payment of the necessary fee.

The invention is described with reference to the several figures of the drawing, in which,

FIG. 1A is a schematic of a polymer microarray produced according to an embodiment of the invention;

FIG. 1B is a schematic of a polymer microarray produced according to an alternative embodiment of the invention;

FIG. 2 is a photomicrograph of an acrylate monomer array deposited on an uncoated glass slide;

FIG. 3A is a photomicrograph of mesenchymal stem cells seeded onto a polymer microarray produced according to an embodiment of the invention; and

FIG. 3B is a enlarged view of a portion of the micrograph in FIG. 3A.

### DETAILED DESCRIPTION

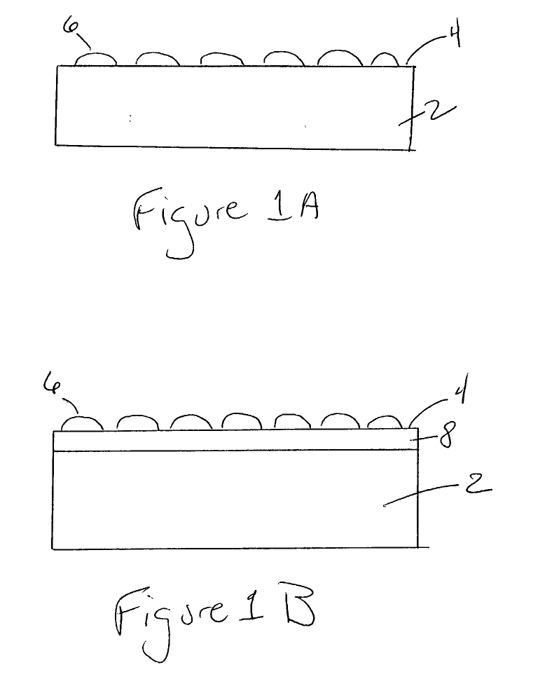

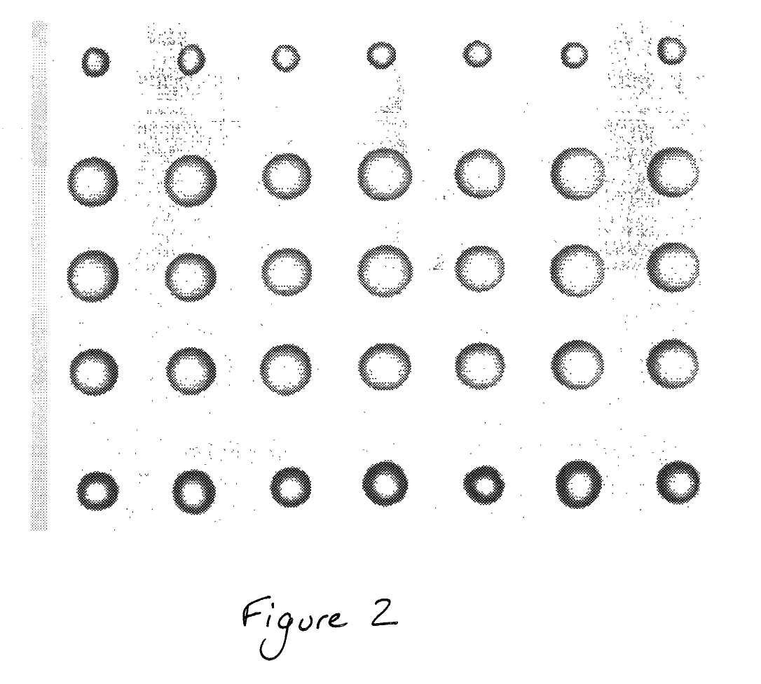

In one embodiment, the microarray of polymers of the present invention comprises a base 2 that is optionally treated to produce a substrate surface 4 across which are dispersed at regular intervals polymer elements 6 (FIG. 1A). The polymer elements 6 are produced by depositing an array of monomers and then polymerizing them in situ. The polymer elements 6 are preferably associated with the substrate surface 4 via non-covalent interactions such as chemical adsorption, hydrogen bonding, surface interpenetration, ionic bonding, van der Waals forces, hydrophobic interactions, magnetic interactions, dipole-dipole interactions, mechanical interlocking, and combinations of these; however, the polymer elements 6 may also be associated with the substrate surface 4 via covalent interactions. The base 2 can be a glass, plastic, metal, or ceramic, but can also be made of any other suitable material. The substrate should be chosen to maximize adherence of the polymer elements while controlling spreading of the deposited monomer. In one embodiment, the surface of the base 2 is rectangular in shape, with dimensions of about 25 mm by 75 mm, and the base is 1 mm thick; however, the base 2 can be of any shape, and may be larger, smaller, thinner or thicker, as chosen by the practitioner. As used herein, the term “substrate” refers to the material on which the polymer elements are dispersed. The substrate may be an uncoated base 2 or include a buffer layer 8 interposed between the base 2 and the polymer elements 6 (FIG. 1B).

For example, the buffer layer 8 may be a polymer. Practically any polymer may be used as a buffer layer. The monomer deposited over the polymer preferably penetrates some distance into such a buffer layer 8. When the monomer is polymerized, the resulting polymer is entangled in the buffer layer 8, increasing adhesion of the polymer element through mechanical interlocking. The degree of entanglement will be determined partially by the depth to which the monomer solutions penetrate after deposition. However, even if the solvent of the monomer stock solution is not miscible with the polymer of the buffer layer 8, the monomer may still be soluble in aqueous media and diffuse from the solvent into the buffer layer 8. After initiation, the polymer chain will form inside the buffer layer 8, creating a mechanical interlock retaining the polymer element 6 on the substrate. In another embodiment, the strands of the polymer element form cross-links with the polymer during polymerization. Of course, even if there is no mechanical or covalent retention of the polymer on the buffer layer, the polymer elements 6 may interact with the buffer layer 8 non-covalently (e.g., through hydrogen bonds, ionic bonds, van der Waals forces, magnetic interactions, etc., including combinations of these). The composition of the polymer for the buffer layer 8 may be chosen to enhance covalent or non-covalent intersections with the base 2, the polymer elements 6, or both. Alternatively or in addition, the base 2 may be modified to enhance its interaction with the polymers of the buffer layer 8. An example of a modified base would be an epoxy modified glass, for example, a light microscope slide or coverslip (e.g., XENOSLIDE™ E available from Xenopore Corp. of Hawthorne, N.J.).

In one embodiment, the polymer buffer layer 8 forms a hydrogel. A hydrogel is defined as a substance formed when an organic polymer (natural or synthetic) is cross-linked via covalent, ionic or hydrogen bonds to create a three-dimensional open-lattice structure that entraps water molecules to form a gel. If cells are to be seeded on the elements 6 of the array, the hydrogel is preferably cytophobic, helping to confine cells seeded onto the microarray to the polymer elements 6. A variety of hydrogels that have a low cell binding affinity are known in the art. In general, these polymers include unsaturated hydrocarbons and polar but uncharged groups, and are at least partially soluble in water or aqueous alcohol solutions. Examples of polymeric hydrogels that have a low cell binding affinity and may be used in the present invention include but are not limited to homopolymers and copolymers of methacrylic acid esters (e.g., polyHEMA), alkylene oxides, and alkylene glycols.

The base 2 may be coated with the polymer buffer layer 8 by dip coating, spray coating, brush coating, roll coating, or spin casting. For example, the base 2 may be coated with the polymer buffer layer 8 by dipping the base 2 in a solution of the polymer. Depending on the composition of the base and the polymer, either an organic or an aqueous solution of the polymer may be employed. In all of the above processing approaches, a suitable cross-linking agent may be incorporated to enhance the mechanical rigidity of the polymer. Divinyl benzene (DVB) and ethylene glycol dimethacrylate (EDMA) are non-limiting examples of cross-linking agents that could be used to crosslink the polymer chains of a hydrogel-forming polymer. The polymer may also be coated on the base as a thin film of oligomers by radiofrequency (RF) plasma deposition. RF plasma deposition is a one step gas phase (i.e., dry) process and is reviewed in great detail in Ratner et al., J. Molec. Recogn. 9:617, 1996; Chinn et al., J. Tiss. Cult. Method. 16:155, 1994; Heshmati et al., Colloque de Physique 4:285, 1990; and Ratner et al., in Plasma Deposition, Treatment and Etching of Polymers, Ed. by R. d'Agostino, Academic Press, San Diego, Calif., 1990; all of which are incorporated herein by reference. As described in Lopez et al., J. Biomed. Mater. Res. 26:415, 1992, RF plasma deposition can, for example, be used to deposit oligomers such as triethylene glycol dimethyl ether or tetraethylene glycol dimethyl ether to form thin poly(ethylene oxide)-like thin films. One skilled in the art will recognize how to apply this technique to a wide variety of polymers. The polymer surface may be modified, for example, with an acid wash or an amine, before deposition of the monomer array.

In another embodiment, the surface of the base is modified to provide a desired surface chemistry. For example, Xenopore Corp. (Hawthorne, N.J.) produces glass slides that are modified to produce a variety of surfaces, including aminosilane, aldehyde, epoxy, maleimide, and thiol. The polymer elements may be produced directly on the surface modified slide, or the slide may be further treated. For example, an acrylate group may be attached to the surface. Amine groups are easily conjugated with a variety of functionalities. Alternatively, an epoxide modified slide may be derivatized or reacted to form a surface having the desired chemistry. Alternatively, a metallic substrate may be treated to provide a specific oxide or sulfide scale. The modified base may be optimized to enhance covalent or non-covalent interactions with a buffer layer or the elements of the microarray. Where a desired property of the substrate, such as conductivity, is anisotropic, the metallic substrate may be textured to control the magnitude of the property. The surface of the base may also be patterned to prevent chemical communication between the polymer elements.

Once the substrate surface of the microarray of the invention has been provided, it will be appreciated by one of ordinary skill in the art that a variety of monomers can be utilized to form the polymer elements of the microarray. In one embodiment of the present invention, liquid monomers are diluted with 25% dimethylformamide (DMF) by volume to decrease viscosity. One skilled in the art will realize that the viscosity may be modified by changing the amount or chemical properties of the solvent. Examples of solvents that may be used to prepare the stock solutions of the present invention include but are not limited to dimethylformamide, dimethylsulfoxide (DMSO), chloroform, dichlorobenzene, and other chlorinated solvents.

In an alternative embodiment, the invention may be practiced with solid monomers. Such monomers should be dissolved in a solvent to be deposited on the substrate. The concentration of the solution is preferably between about 1M and about 3M. More dilute solutions than 1M may be used, but it may be necessary to evaporate excess solvent after polymerization. It may be necessary to use a less volatile solvent, such as DMSO, to dissolve the solid monomers to prevent the solvent from evaporating before polymerization or to provide the proper viscosity of the stock solution. Alternatively, as discussed below, the microarray may be deposited and polymerized in small sections.

In one embodiment, the monomer is part of a biocompatible polymer. A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference). Exemplary biocompatible polymer classes that may be incorporated into polymer elements 6 using the techniques of the invention include polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, poly(vinyl chloride), poly(vinyl fluoride), poly(vinyl imidazole), poly(vinyl alcohol), and chlorosulphonated polyolefins. The term biodegradable, as used herein, refers to materials that are enzymatically or chemically (e.g., hydrolytically) degraded in vivo into simpler chemical species. Monomers that are used to produce these polymers are easily purchased from companies such as Polysciences, Sigma, Scientific Polymer Products, and Monomer-Polymer & Dajac Laboratories. These monomers may be combined in an array to form a wide variety of co-polymers.

Preferably, the monomers polymerize by chain polymerization. Exemplary monomers subject to radical chain polymerization include ethylene, vinyl derivatives of ethylene, including but not limited to vinyl acetate, vinyl chloride, vinyl alcohol, and vinyl benzene (styrene), vinylidine derivatives of ethylene, including but not limited to vinylidine chloride, acrylates, methacrylates, acrylonitriles, acrylamides, acrylic acid, and methacrylic acid, fluoropolymers, dienes, including but not limited to butadiene, isoprene, and their derivatives, and aromatic monomers such as phenylene and its derivatives, such as phenylene vinylene. Monomers such as α-olefins, 1,1-dialkyl olefins, vinyl ethers, aldehydes, and ketones may be polymerized by anionic chain polymerization, cationic chain polymerization, or both. Additional monomers can be found in George Odian's Principles of Polymerization, (3rd Edition, 1991, New York, John Wiley and Sons), the entire contents of which are incorporated herein by reference.

One skilled in the art will recognize that the techniques of the invention may also be exploited to produce microarrays by step polymerization. The reaction conditions for a variety of polyesters, polyamides, polyurethanes, and other condensation polymers are well known in the art (see Odian, 1991). Such reactions may be easily adapted to produce microarrays on substrates. In one embodiment, neat monomers are deposited as a liquid or in a solution with a solvent such as DMSO or chloroform to prevent premature precipitation of the polymer. Non-volatile solvents are preferred to reduce evaporation. A catalyst, for example, sulfuric acid or p-toluenesulfonic acid, may be used to increase the rate of reaction. The substrate may be heated or placed in a low pressure atmosphere to drive off the condensation product and drive the reaction. The low volume and high surface area of the droplets should facilitate the removal of the condensation product without the use of purging gases or high vacuum conditions.

Monomers that require chemical initiators may also be used. If the initiator works at a specific temperature, the monomer solutions should be cooled during deposition and then warmed to initiate polymerization. It may be desirable to use a less viscous solvent than would be employed to deposit the microarray at room temperature. In an alternative embodiment, monomers may be deposited in a microarray and then exposed to an ozone atmosphere to initiate polymerization.

The molecular weight of the resultant polymer may be controlled by adjusting the properties of the solvent. Modifying the viscosity of the solvent changes the polymerization rate and the resulting molecular weight distribution. Some solvents provide a more favorable environment for radicals and intermediate products formed during polymerization and allow polymerization to continue for a longer time before termination. The selection of solvents to stabilize or destabilize radicals or to promote condensation and other step polymerization reactions is well known to those skilled in the art.

In an alternative embodiment, the molecular weight of the polymer may be controlled by varying the concentration of monomer in the stock solution or the ratios of difunctional monomers to unifunctional monomers. Increased concentrations of difunctional monomers will increase the degree of cross-linking in the chains. Monofunctional monomers may be modified to form difunctional monomers by reacting them with a linker chain. Appropriate linkers and chemical reactions will be evident to one skilled in the art. For example, dicarboxylic acids are reactive with a wide variety of functional groups commonly incorporated into vinyl monomers, including alcohols, amines, and amides.

In one embodiment, diacrylate monomers are used to produce the polymer arrays of the invention. Diacrylates are available as liquids and are easily polymerized upon exposure to UV light. Exemplary diacrylate monomers for use with the invention are listed in Table 1. Preferably, these monomers are diluted by 25% with DMF before spotting to reduce their viscosity and ensure reproducible deposition onto the substrate. One skilled in the art will recognize that mixtures of diacrylate and monoacrylate monomers may be used to control the degree of cross-linking in the polymer.

TABLE 1

| Diacrylate species | Source |
|---|---|
| 1,4 butanediol dimethacrylate | Scientific Polymer Products |
| diethylene glycol diacrylate | Scientific Polymer Products |
| diethylene glycol dimethacrylate | Scientific Polymer Products |
| 1,6 hexanediol diacrylate | Scientific Polymer Products |
| neopentyl glycol diacylate | Scientific Polymer Products |
| phenylene diacrylate 1,3 | Polysciences |
| propoxylated neopentyl glycol diacrylate | Scientific Polymer Products |
| tetraethylene glycol diacrylate | Scientific Polymer Products |
| tetraethylene glycol dimethacrylate | Scientific Polymer Products |
| triethylene glycol diacrylate | Scientific Polymer Products |
| triethylene glycol dimethacrylate | Scientific Polymer Products |
| tripropylene glycol diacrylate | Scientific Polymer Products |
| caprolactone 2-(methacryloyloxy)ethyl ester | Sigma |
| 5-ethyl-5-(hydroxymethyl)-β,β-dimethyl-1, | Sigma |
| 3-dioxane-2-ethanol diacrylate | |
| 1,6-hexanediol propoxylate diacrylate | Sigma |
| 3-hydroxy-2,2-dimethylpropyl 3-hydroxy-2, | Sigma |
| 2-dimethylpropionate diacrylate | |
| glycerol 1,3-diglycerolate diacrylate | Sigma |
| glycerol dimethacrylate, mixture of isomers, | Sigma |
| tech. | |
| 85%, neopentyl glycol dimethacrylate | Sigma |
| neopentyl glycol ethoxylate (1 EO/OH) | Sigma |
| diacrylate | |
| trimethylolpropane benzoate | Sigma |
| diacrylate | |
| 1,14-tetradecanediol dimethacrylate | Sigma |
| tricyclo[5.2.1.0^{2,6}]decanedimethanol | Sigma |
| diacrylate | |
| trimethylolpropane ethoxylate (1 EO/OH) | Sigma |
| methyl ether diacrylate | |
| trimethylolpropane triacrylate, tech. | Sigma |

In an alternative embodiment, additional chemical species may be incorporated into the polymers of the invention. For example, as is well known in the art, the attachment, growth and differentiation of cells on synthetic polymers may be enhanced by incorporating certain natural compounds with the synthetic polymers. These include but are not limited to polypeptides and polypeptide derivatives such as glycoproteins, lipoproteins, hormones, antibodies, basement membrane components (e.g., laminin, fibronectin), collagen types I, II, III, IV, and V, albumin, gelatin, fibrin, and polylysine; polysaccharides and polysaccharide derivatives such as agar, agarose, gum arabic, and alginate; glycosaminoglycans such as heparin, heparin sulfate, chondroitin, chondroitin sulfate, dermatin, and dermatin sulfate; and polynucleotides such as genes, antisense molecules which bind to complementary DNA to inhibit transcription, ribozymes and ribozyme guide sequences. Natural compounds for use with the invention may also include immunomodulators, inhibitors of inflammation, regression factors, inducers of differentiation or de-differentiation, attachment factors, growth factors, and lipids. Examples of growth factors that may be used in the present invention include but are not limited to heparin binding growth factor (HBGF), alpha or beta transforming growth factor (α- or β-TGF), alpha fibroblastic growth factor (α-FGF), epidermal growth factor (EGF), vascular endothelium growth factor (VEGF), nerve growth factor (NGF) and muscle morphogenic factor (MMP). Examples of lipids that may be used in the present invention include but are not limited to L-alpha-phosphatidyl-L-serine, L-alpha-phosphatidyl-DL-glycerol, L-alpha-phosphatidic acid, L-alpha-phosphatidylcholine, L-alpha-lysophosphatidylcholine, sphingomyelin, and cardiolipin. Such compounds are well known in the art and are commercially available or described in the controlled drug delivery or tissue engineering literature.

Synthetic compounds may also be incorporated into polymer elements 6 produced according to the invention. Examples of synthetic biologically active compounds that can be present as components of polymeric materials of the microarray of the invention include but are not limited to drugs and combinatorial compounds. For example, one particularly attractive application of the present invention would involve using a microarray of polymeric biomaterials according to the present invention to screen the compounds of a combinatorial library for novel effects on cellular behavior. In one embodiment, the compounds are drugs that have already been deemed safe and effective for use by the appropriate governmental agency or body. For example, drugs for human use listed by the Food and Drug Administration (FDA) under 21 C.F.R. §§ 330.5, 331-361, 440-460, and drugs for veterinary use listed by the FDA under 21 C.F.R. §§ 500-582, all of which are incorporated herein by reference, are all considered acceptable for use in the present inventive microarray. A more complete non-limiting listing of classes of synthetic compounds suitable for use in the present invention may be found in the Pharmazeutische Wirkstoffe Ed. by Von Kleemann et al., Stuttgart/New York, 1987, incorporated herein by reference.

In one embodiment, the techniques of the invention may be used to create libraries of biological compounds for in vitro testing. Arrays of peptides may be tested for their interactions with cells. DNA arrays may be tested against known complementary strands and the degree of hybridization measured. The effect of potential drugs on cells and biological molecules may also be investigated. Thus, the techniques of the invention may be used not only to test polymer compositions but to immobilize other libraries of compounds.

While the advantages of the invention are easily recognized in the biomedical sciences, other fields may also benefit from the teachings of the invention. For example, many electrical and mechanical applications do not require biocompatible polymers. Easily purchased monomers such as diols, diacids, diamines, diisocyanates, vinyl compounds, dienes, substituted alkynes, aldehydes, and ketones may be combined to form polymer elements in the arrays of the invention (see the Aldrich catalog, 2000-2001 edition, available from Sigma-Aldrich, Milwaukee, Wis., the contents of which are incorporated herein by reference). Combinations of monomers that form polymers with conjugated backbones, such as aniline, may be used to produce polymers having different electrical conductivities. Highly conjugated polymers may also be tested for charge storage.

Non-biologically active materials may also be incorporated into the polymer elements 6. For example, a chelating agent may be functionalized and incorporated into the polymer. After polymerization, a ligand such as platinum may be immobilized on the polymer element and used as a catalyst. Magnetic particles may be incorporated into the polymer in the same manner. Conducting or luminescent materials may be used to formulate molecularly doped polymers. Instead of varying the composition of the polymer, the composition or amount of dopant may be varied across the array to identify materials and compositions for sensors and other devices. For example, a microarray formed according to the invention may be placed in a controlled environment and the response of the dopant to various partial pressures of oxygen measured. Dyes may also be exploited as dopants. Because the thickness of the spot may change the wavelength of the output, precise control of monomer deposition, e.g., by an ink-jet printer, is preferred where dyes are used.

The compound or compounds to be added to the polymer may be functionalized to contain a moiety that is incorporated into the polymer and mixed with some or all of the monomers being deposited. For example, a vinyl group, an acrylamide, an epoxide, or an acrylate group is attached to the compound. During polymerization, the functionalized compounds are incorporated into the polymer without post-polymerization chemistry. For example, an amino acid sequence such as RGD or an antibody may be attached to a carboxyl group in an acrylate or diacrylate monomer. Exemplary methods of attaching acrylate groups to biomolecules are disclosed in Burkoth, et al., “Surface and bulk modifications to photocrosslinked polyanhydrides to control degradation behavior,” J. Biomed. Mater. Res., 2000, 21:2395, and Hern, et al., “Incorporation of adhesion peptides into nonadhesive hydrogels useful for tissue resurfacing,” J. Biomed. Mater. Res., 1998, 39:266-276, the entire contents of both of which are incorporated herein by reference. A heterobifunctional cross-linking agent, e.g., from Pierce Biotechnology (Rockford, Ill.) will present both an appropriate group for derivatizing a biomolecule or other additive and a functional group that can be incorporated into a polymer backbone. In an alternative embodiment, the moiety with which the compound is functionalized includes a photoreactive group that can initiate polymerization when exposed to UV light. These compounds may also be attached to a difunctional monomer for step polymerization or derivatized with the appropriate functional groups, e.g., a pair of amines or carboxylate groups, and incorporated into the polymer backbone.

Alternatively, additional chemical species may be attached to the species incorporated into the polymer, with the incorporated chemical species serving as a linker. As noted above, chelating agents may be used to incorporate metals into the polymer. This technique may be used to incorporate other chemical species into the polymer as well. For example, the invention may employ a ligand/receptor type interaction to indirectly link a biological compound and a synthetic polymer of the invention. Any ligand/receptor pair with a sufficient stability and specificity to operate in the context of the inventive system may be employed. To give but one example, the compound may be linked or associated with biotin and avidin (or streptavidin) incorporated into the polymer. For example, the biotin, etc. may be functionalized as a monomer. The strong binding of biotin to avidin (or streptavidin) would then allow for association of the compound with the synthetic polymer. Other possible ligand/receptor pairs include antibody/antigen, FK506/FK506-binding protein (FKBP), rapamycin/FKBP, cyclophilin/cyclosporin, and glutathione/glutathione transferase pairs. Other ligand/receptor pairs are well known to those skilled in the art.

Once the appropriate monomer and the substrate surface have been selected for use in the present invention, it will be appreciated that the monomers can be formed into a polymer microarray on the substrate surface using a range of techniques known in the art. In one embodiment of the present invention, the elements of the microarray are formed by depositing small drops of each monomer solution at discrete locations on the substrate surface, preferably by using an automated liquid handling device. As mentioned above, the monomers of the invention are initially provided as diluted liquids or solutions of dissolved solids. Once the stock solutions of the polymeric biomaterials have been prepared, a predetermined volume of each biomaterial stock solution is placed in the separate reservoirs of the robotic liquid handling device.

The drops may be deposited on the substrate surface using a microarray of pins (e.g., ChipMaker2™ pins, available from TeleChem International, Inc. of Sunnyvale, Calif.). A range of pins exist that take a sample volume up by capillary action and deposit a spot volume of 1 to 10 nl or more. In another embodiment, the drops may be deposited on the substrate surface using syringe pumps controlled by micro-solenoid ink-jet valves that deliver volumes greater than about 10 nl (e.g., using printheads based on the SYNQUAD™ technology, available from Cartesian Technologies, Inc. of Irvine, Calif.). Alternatively, the drops may be deposited on the substrate surface using piezoelectric ink-jet fluid technology that deposits smaller drops with volumes between about 0.1 and 1 nl (e.g., using the MICROJET™ printhead available from MicroFab Technologies, Inc. of Plano, Tex.). Alternative techniques may be employed to deposit smaller or larger drops. For example, pins may be pre-tapped to release a large drop and then tapped on the substrate to release a smaller drop, just as a paintbrush is tapped on the side of the can to remove excess paint and prevent messy drips on the painted surface. Where small drops are used, they should be polymerized shortly after deposition, before the solvent evaporates. For example, a portion of an array may be deposited and polymerized before deposition of a second portion of the array.

In one embodiment, the drops are arranged as a rectangular microarray on a glass slide. The size of the array may be determined by the user and will depend on the size of the elements of the array, the spacing between the elements and the size of the substrate surface. The rectangular microarray may, for example, be an 18×40, an 18×54 or a 22×64 microarray; however, smaller, larger and alternatively shaped microarrays (e.g., square, triangular, circular, elliptical, etc.) may be used. The shape of the microarray and the arrangement and spacing of polymer elements within it may depend on the analytical methods used to examine the arrayed polymers. For example, a particular sensor may require a specific shape or distribution of polymer elements. Fluidic testing of the polymers may require a specific arrangement and spacing of polymer elements. One skilled in the art will recognize that the use of robotic controls to move the pins enables any distribution and arrangement of spots regardless of symmetry. In one embodiment, two or more identical arrays are deposited alongside one another so that experiments on the polymers may be repeated.

In one embodiment of the invention, each element of the microarray is formed by depositing a single drop taken from one of the monomer stock solutions. In another embodiment, some or all of the elements are formed by depositing at least two drops taken from one of the monomer stock solutions. In yet another embodiment, some or all of the elements are formed by depositing at least two drops taken from at least two different monomer stock solutions. It may be advantageous to layer the same or different monomers on a single element of the microarray by polymerizing a spot before depositing another drop on the same element. For example, one could bury a polymer layer of interest within several biodegradable layers so that access to the layer of interest, or alternatively, release of a compound from the layer of interest can be controlled. The use of biodegradable polymers for this purpose is well known in the art of tissue engineering and drug delivery.

Preferably, the dimensions of the elements of the microarray are substantially the same; however, in certain embodiments of the present invention, the dimensions of the elements of the microarray may differ from one element to the next. The “vertical dimension”, as that term is used herein, means the vertical dimension of the element when viewed from a direction that is parallel to the substrate surface (i.e., from the side). The “horizontal dimension”, as that term is used herein, means the horizontal dimension of the element when viewed from a direction that is perpendicular to the substrate surface (i.e., from above).

The vertical dimensions of elements of the microarray of the present invention are such that each element may comprise hundreds or even thousands of layers of polymer molecules. When viewed from above or from the side, the elements may be circular, oblong, elliptical, square or rectangular. Preferably, the overall shape of the elements is sphere-like or disk-like. In one embodiment, the drops are deposited at intervals that range from about 300 to about 1200 μm. In a preferred embodiment, the drops are deposited at about 720 μm intervals; however, the drops may be deposited at smaller or larger intervals. The size and density of the elements depends on the application. Smaller elements, e.g., spaced at intervals of 1 μm or less, may be preferred for chemical analysis to further increase the number of compounds that can be analyzed in one batch. For example, 100 million elements, spaced at 0.1 μm intervals, can fit in an area of a square millimeter. In other embodiments, the array may have a density of one or fewer polymer elements per square centimeter. In general, the density, vertical dimension, and horizontal dimension of the elements will be optimized for the particular manufacturing technique and the variable being tested.

In an exemplary embodiment of the invention, the elements of the microarray are deposited on the substrate surface as drops that range in volume from 0.1 to 100 nl. However, smaller and larger volumes may be deposited on the substrate surface. The ultimate dimensions of the drops depend on the application. For example, for cell attachment, the vertical dimension of the elements should be between about 50 and 500 μm, and the horizontal dimension of the deposited drops should be between 300 and 600 μm. The element should be large enough to minimize edge effects, but, for a single cell, the element may not need to be any larger than 10 μm across. The drop volume and monomer viscosity may be adjusted so that the polymer element is thinner than 50 μm or even essentially flat. The primary limits on drop size are the ability to detect and deposit tiny drops. For some applications, it may be desirable to deposit drops as thin as a few 10 s of nanometers. Microinjectors and robots can produce arrays of miniscule droplets, but the viscosity of the precursor must be carefully controlled to prevent clogging. Ink-jet printers may be used to reproducibly deposit drops of a specified size. In addition, the precursor should not polymerize before deposition and perhaps clog the dispenser. Thicker polymer elements may be produced by depositing a larger volume of precursor solution or by depositing several layers at each location. Bigger drops are easily deposited by e.g., using bigger pins (e.g., from TeleChem International, Inc., Sunnyvale, Calif.).

Pre- and post-deposition processing may also be used to control the size of the polymer elements. Polymerized elements may be etched, ground, smoothed, or partially melted to reduce their thickness. Before deposition, the surface of the base may be modified to increase wetting, spreading a polymer element and decreasing its vertical dimension without changing its volume. Alternatively, a monomer may be mixed with a greater amount of solvent or a less viscous solvent to increase spreading. Portions of the surface may be modified to add an additional variable to an array, or several formulations of the same monomer may be used, or some combination of these techniques. A wide variety of properties vary with thickness, including conductivity, luminescence, and various mechanical properties.

After the monomer has been deposited on the surface, it is polymerized. In a preferred embodiment, e.g. of diacrylates, the microarray is exposed to UV light, which initiates polymerization. If a chemical initiator is used, the microarray is exposed to conditions under which the initiator will start reacting with the monomer. Exemplary radical initiators that may be used with the invention include, but are not limited to, azobisisobutylnitrile (AIBN), 2,2-dimethoxy-2-phenyl-acetophenone (DPMA), benzoyl peroxide, acetyl peroxide, and lauryl peroxide. Redox and thermal initiators may also be exploited. For example, peroxides may be combined with a reducing agent such as Fe²⁺, Cr²⁺, V²⁺, Ti³⁺, Co²⁺, Cu⁺, and amines such as N,N-dialkylaniline. These initiators may be mixed with the monomer solutions and co-deposited. Because such initiators are often sensitive to temperature, they should be deposited at depressed temperatures. The temperature is then raised to start polymerization. A monomer that polymerizes in air should be deposited under nitrogen or argon and then exposed to air to start polymerization. One skilled in the art will recognize that a wide variety of initiators may be employed with the invention depending on the monomes being deposited. A plethora of initiators are available from companies such as Sigma and Polysciences. In one embodiment of the invention, once the complete microarray of elements has been deposited and polymerized, the polymer microarray is placed in an evacuated desiccator at about 25° C. for 12 to 48 hrs to remove any residual solvent. Alternatively, or additionally, the microarray may be washed to remove the solvent.

Polymers may be layered in the elements of the microarray by depositing additional monomer on the polymerized microarray and polymerizing it. The first layer of the polymer may become more highly cross-linked. Alternatively, this may be avoided by using monomers that are initiated under different conditions, for example, UV light and air. The interface of the two polymer layers will not be discrete. The monomer of the second layer may penetrate some distance into the first layer, resulting in interweaving of the polymer chains. In addition, the monomer of the second layer may react with unreacted monomer or chain ends in the first layer, providing a covalent link between the two layers.

In one embodiment, the substrate surface 4 or the array is modified after the polymer array has been deposited. Self assembled monolayer (SAM) systems may be chosen that react with the base layer but not with the various polymers. Alternatively, the polymer array may be deposited directly on the substrate and the uncovered surface modified afterwards using standard organosilane chemistry. For example, it is well known that washing PLGA in an acidic solution makes it more cytophilic. Both acid and base washes may be tested on other polymers. Alternatively, the droplets may be coated by dipping them into solutions of materials like fibronectin or plating solutions.

One aspect of the present invention involves the recognition that an endless variety of polymers and combinations of polymers with natural and/or synthetic compounds can be obtained according to the present invention by varying the compositions of the stock solutions that are initially added to the robotic liquid handling device and/or by layering drops taken from these stock solutions in a series of sequential deposition steps. To produce bulk quantities of polymers would require large amounts of monomer and solvents which would then have to be disposed of properly. Small amounts of stock solutions of the desired monomers can be used for multiple tests, enabling a large number of monomers to be mixed in several different proportions in a single experiment. In addition, fewer stock solutions are required than to deposit polymerized polymers in the array.

The invention not only enables the practitioner to produce immobilized combinatorial libraries of polymers but to immobilize libraries of other compounds in an array. For example, combinatorial chemistry may be used to produce a library of acrylate-functionalized molecules. These molecules are then mixed into a series of stock solutions of an acrylate monomer and polymerized in an array according to the techniques of the invention.

The composition of the polymers themselves may be analyzed spectrophotometrically, for example, by fluorescence, infrared, or Raman spectroscopy. In an exemplary embodiment, the optical properties of the polymers are identified. For example, light emitting polymers may prove useful for LEDs. Alternatively, polymers may be tested for their ability to immobilize a photoactive species. For example, an array of polymers may be functionalized with different antibodies. The array is treated with antigens derivatized with a photoactive species, which is then localized on the surface.

In one embodiment of the present invention, a microarray of biocompatible polymers provided according to the invention may be seeded with cells. The invention employs a wide range of cell types and is not limited to any specific cell type. Examples of cell types that may be used include but are not limited to bone or cartilage forming cells such as chondrocytes and fibroblasts, other connective tissue cells such as epithelial and endothelial cells, cancer cells, hepatocytes, islet cells, smooth muscle cells, skeletal muscle cells, heart muscle cells, kidney cells, intestinal cells, other organ cells, lymphocytes, blood vessel cells, and stem cells such as human embryonic stem cells or mesenchymal stem cells. For therapeutic applications, it is preferable to practice the invention with mammalian cells, and more preferably human cells. However, non-mammalian cells such as bacterial cells (e.g., E. coli), yeast cells (e.g., S. cerevisiae) and plant cells may also be used with the present invention.

The cells are first cultured in a suitable growth medium as would be obvious to one of ordinary skill in the art. See, for example, Current Protocols in Cell Biology, Ed. by Bonifacino et al., John Wiley & Sons Inc., New York, N.Y., 2000 (incorporated herein by reference). A microarray of biocompatible polymers prepared as above is then placed in a suitable container (e.g., a 25 mm by 150 mm round suspension culture dish) and incubated with a solution of the cultured cells. Preferably the cells are present at a concentration that ranges from about 10,000 to 500,000 cells/cm³, although both higher and lower cell concentrations may be used. The incubation time and conditions (e.g., temperature, CO₂ and O₂ levels, growth medium, etc.) will depend on the nature of the cells that are under evaluation. For most cell types, the choice of conditions will be obvious to one skilled in the art. The incubation time should be sufficiently long to allow the cells to adhere to the elements of the polymeric biomaterial microarray. In one embodiment of the invention, the environmental conditions will need to be optimized in a series of screening experiments.

In an alternative embodiment, fully differentiated cells are seeded onto the microarray. Once the cells have been incubated for an appropriate amount of time, stem cells are seeded onto the microarray. The same stem cells may be used for every element, or different stem cells may be used to create a library of combinations of differentiated cells and stem cells. Stem cells appropriate for use with the invention include embryonic stem cells, mesenchymal stem cells, and progenitor cells for tissues such as bone and liver. The influence that each cell type has on the other's behavior is then assayed using the techniques described below.

In a preferred embodiment of the invention, the cellular behavior of the seeded cells is assayed for each element of the microarray. The invention employs a wide range of cell-based assays that enable the investigation of a variety of aspects of cellular behavior. Exemplary cell-based assays are discussed in our commonly owned application U.S. Ser. No. 09/803,319, entitled “Uses and Methods of Making Microarrays of Polymeric Biomaterials,” the entire contents of which are incorporated herein by reference.

The cellular behaviors that can potentially be investigated according to the invention include but are not limited to cellular adhesion, proliferation, differentiation and gene expression. One may be interested in screening for polymeric biomaterials that promote or inhibit the adhesion of a given cell type. It is also desirable to understand whether certain materials are toxic to cells or accelerate apoptosis. Alternatively or additionally, one may be interested in screening for biocompatible polymers that enhance the proliferation of a given cell type. For example, biocompatible polymers that enhance the adhesion and proliferation of chondrocytes could be used as scaffolds in the preparation of engineered cartilage. One may further be interested in screening for polymeric biomaterials that cause attached cells to differentiate or de-differentiate in a desirable way. More specifically, one may be interested in screening for polymeric biomaterials that promote or inhibit the expression of a given gene within a cell. For example, polymeric biomaterials that support differentiation of neural stem cells into glial cells or neurons may be useful as scaffolds in the regeneration of neural tissue. Different growth factors or growth media may be tested to enhance this effect. Alternatively, it may be desirable to characterize the influence of a polymer on a cell's interaction with other cells, viruses, small molecules, DNA, biomolecules, etc. The cell's interactions with a selection or library of chemicals may be evaluated by producing an array with one polymer on which a variety of small molecules, DNA, biomolecules, etc. are immobilized.

It will be appreciated that any of the cell-based assays known in the art may be used according to the present invention to screen for desirable interactions between the biocompatible polymers of the microarray and a given cell type. When they are assayed, the cells may be fixed or living. Preferred assays employ living cells and involve fluorescent or chemiluminescent indicators, most preferably fluorescent indicators. A variety of fixed and living cell-based assays that involve fluorescent and/or chemiluminescent indicators are known in the art. For a review of cell-based assays, see Current Protocols in Cell Biology, Ed. by Bonifacino et al., John Wiley & Sons Inc., New York, N.Y., 2000; Current Protocols in Molecular Biology, Ed. by Ausubel et al., John Wiley & Sons Inc., New York, N.Y., 2000; Current Protocols in Immunology, Ed. by Coligan et al., John Wiley & Sons Inc., New York, N.Y., 2000; Sundberg, Curr. Opin. Biotechnol. 11:47, 2000; Stewart et al., Methods Cell Sci. 22:67, 2000; and Gonzalez et al., Curr. Opin. Biotechnol. 9:624, 1998; all of which are incorporated herein by reference.

Specific cell-based assays that can be used according to the present invention include but are not limited to assays that involve the use of phase contrast microscopy alone or in combination with cell staining; immunocytochemistry with fluorescent-labeled antibodies; fluorescence in situ hybridization (FISH) of nucleic acids; gene expression assays that involve fused promoter/reporter sequences that encode fluorescent or chemiluminescent reporter proteins; in situ PCR with fluorescently labeled oligonucleotide primers; fluorescence resonance energy transfer (FRET) based assays that probe the proximity of two or more molecular labels; and fused gene assays that enable the cellular localization of a protein of interest. The steps involved in performing such cell-based assays are well known in the art.

The polymers synthesized using the techniques of the invention need not be biocompatible. Fields besides biology and tissue engineering can benefit from the ability to test large numbers of polymers quickly. For example, polymers may be tested for their adsorptive selectivity with respect to certain chemicals that are purified by chromatography. For example, an array may be incubated with a solution of a desired chemical. If the chemical is photoactive, it may then be located using the techniques described above. Alternatively, if the chemical has a specific conductivity, then the conductivity of the elements of the array may be measured to locate the chemical. The process can be repeated to determine the adsorption of other chemicals from which the desired chemical will be separated. The selected polymers can then be coated onto silica or glass beads for use in a column.

Alternatively, polymers may be tested for their ability to immobilize a particular catalyst or to catalyze reactions themselves. A practitioner seeking to create a polymer having a specific hydrophilicity or pH may test a range of ratios of certain monomers. The properties of the polymer elements may be tested directly, or a titrant may be added and monitored. The titration end-point may be monitored optically by identifying the point at which the titrant changes color. Alternatively, the end-point may be monitored by tracking the conductivity of the polymer element.

A variety of techniques may be used to test the mechanical properties of polymer spots. Such spots may be contacted at two points and tested using traditional techniques. Alternatively, the spots may be deposited on an elastic or piezoelectric substrate. As the substrate is mechanically stressed, it will exhibit different mechanical behaviors depending on the mechanical properties of the polymer spots. Ultrasound may also be used both to measure the mechanical properties of materials and to exploit known mechanical properties to measure rates of reaction, molecular weight distribution, etc. A nanoindenter may be also be used to measure mechanical properties such as modulus and hardness.

Additional properties that can be tested include, for example, electrical, thermal, mechanical, morphological, optical, magnetic, chemical, etc. Exemplary properties are listed in U.S. Pat. No. 6,045,671, issued Apr. 4, 2000, the entire contents of which are incorporated herein by reference. Any material exhibiting a useful property may be produced in larger quantities for further experiments or incorporation into a device. Exemplary scanning systems that can be used to screen for these properties include, without limitation scanning Raman spectroscopy; scanning NMR spectroscopy; scanning probe spectroscopy including, for example, surface potentialometry, tunnelling current, atomic force, acoustic microscopy, shearing-stress microscopy, ultra-fast photo excitation, electrostatic force microscope, tunneling induced photo emission microscope, magnetic force microscope, microwave field-induced surface harmonic generation microscope, nonlinear alternating-current tunnelling microscopy, near-field scanning optical microscopy, inelastic electron tunneling spectrometer, etc.; optical microscopy in different wavelength; scanning optical ellipsometry (for measuring dielectric constant and multilayer film thickness); scanning Eddy-current microscope; electron (diffraction) microscope, etc.

More particularly, to screen for conductivity and/or superconductivity, one of the following devices can be used: a scanning RF susceptibility probe, a scanning RF/microwave split-ring resonator detector, or a scanning superconductors quantum interference device (SQUID) detection system. To screen for magnetoresistance, a scanning RF/microwave split-ring resonator detector or a SQUID detection system can be used. To screen for crystallinity, infrared or Raman spectroscopy can be used. To screen for magnetic strength and coercivity, a scanning RF susceptibility probe, a scanning RF/microwave split-ring resonator detector, a SQUID detection system or a Hall probe can be used. To screen for fluorescence, a photodetector or a charged-coupled device camera can be used. Additional analysis tools are disclosed in U.S. Pat. No. 6,030,917, the entire contents of which are incorporated herein by reference. Other scanning systems known to those of skill in the art can also be used.

The techniques of the reaction may be used to create arrays of microreactors to perform combinatorial chemistry. Microarrays of polymers whose compositions vary by pH or nucleophilicity may be deposited on a surface that repels the solvent in which the reactants are dissolved, e.g., a hydrophobic substrate is used to test a reaction in an aqueous solvent. The reactants are then deposited on the microarray and allowed to react. Alternatively, the substrate is immersed in a solution containing the reactants, which then form drops on the elements of the microarray by surface tension. The substrate may be heated, placed in a specialized atmosphere, or otherwise processed to facilitate the reaction. The products may be identified using standard spectrophotometric techniques.

In another embodiment, the techniques of the invention may be used to test polymers for specific electrical properties. For example, an array of polymer elements may be sandwiched in between a conductive base layer and a conductive coating. In a preferred embodiment, each polymer element includes several layers of the particular polymer. A voltage is applied between the base layer and the coating and a detector used to identify those polymers which have a band gap with the same or less energy as the applied voltage. Such polymers may be fabricated into voltage specific semiconductors or LEDs. In one embodiment, the base layer is deposited on silica that has been patterned with the appropriate electrical circuit to apply the voltage.

### EXAMPLES

### Example 1

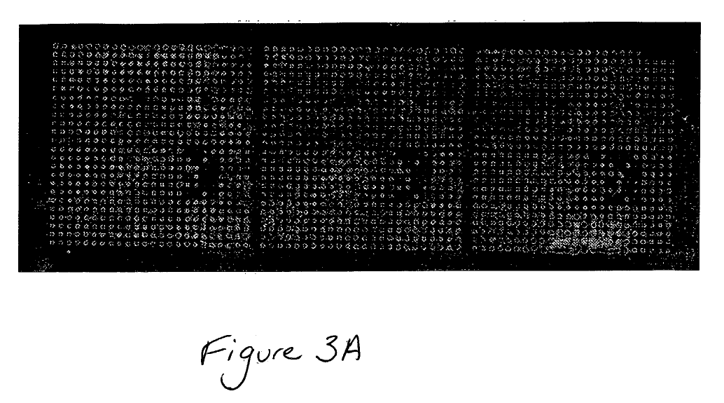

An array of diacrylates was prepared on an uncoated epoxide-modified glass slide supplied by Xenopore. Different sized drops were deposited to form polymer elements having different sizes. The polymer elements are essentially dome shaped (FIG. 2).

### Example 2

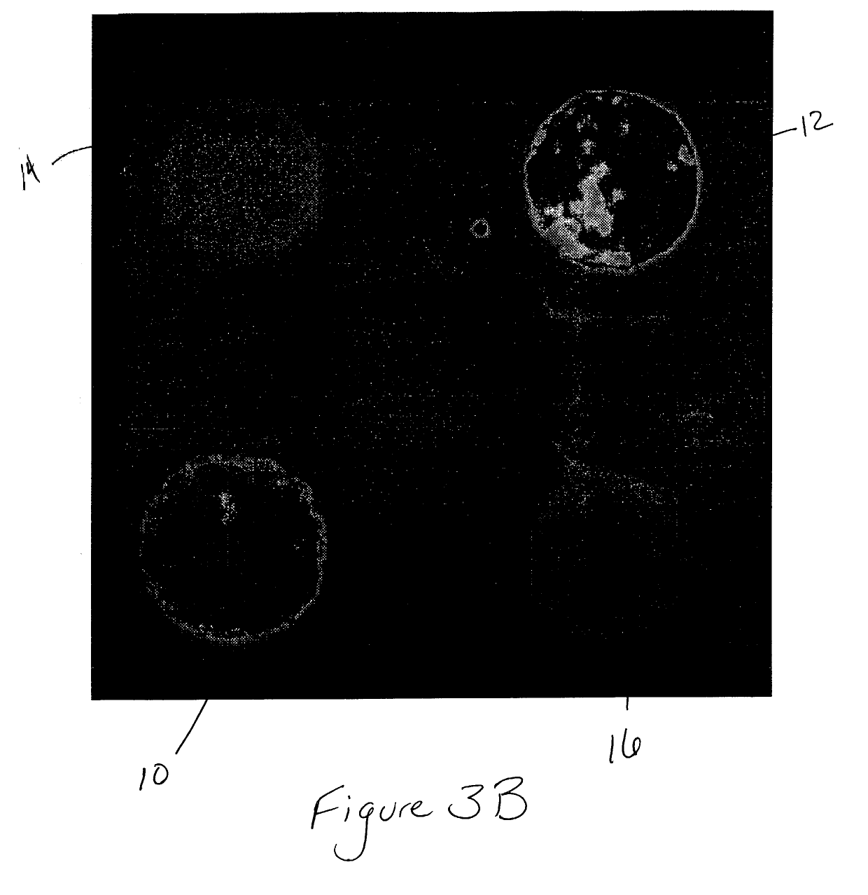

An array of 70/30 (by volume) co-polymers of 24 diacrylates was prepared in triplicate on a epoxide-modified glass slide coated with polyHEMA. The diacrylates were deposited as a grid with the primary (70%) monomer varied along one axis and the 30% monomer varied along the second axis. Thus, the polymer elements along the diagonal of the microarray each include only one monomer species. Human mesenchymal stem cells were seeded onto the microarray, and fluorescence microscopy used to identify those elements to which the cells adhered (FIGS. 3A, 3B). The cells and the polymer luminesce at different wavelengths. The cells exhibit increased contrast with the background. FIG. 3B shows a higher magnification version of a portion of the array shown in FIG. 3A. Cells adhere to elements 10 and 12 but not to 14 and 16.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method of preparing a microarray of polymer elements comprising:
providing a substrate surface;
providing a plurality of individual monomers in a liquid phase;
depositing said monomers as a plurality of discrete monomer elements on said substrate surface; and
exposing the plurality of discrete monomer elements to initiating conditions so that polymer elements are formed.

2. The method of claim 1, wherein at least a portion of the discrete elements include more than one type of monomer.

3. The method of claim 1, wherein at least a portion of the monomers are a liquid at room temperature.

4. The method of claim 3, wherein the liquid is combined with a solvent.

5. The method of claim 1, wherein at least a portion of the monomers are a solid at room temperature and providing the monomers in a liquid phase comprises dissolving the monomers in a solvent at a concentration of about 3M or less.

6. The method of claim 1, wherein the polymer elements are a first portion of the microarray of polymer elements, and the method further comprises repeating the steps of depositing and exposing to prepare a second portion of the array of polymer elements.

7. The method of claim 1, wherein the steps of depositing and exposing comprise:
A) depositing a single discrete monomer element on said substrate surface using a robotic liquid handling device;
B) exposing the discrete monomer element to initiating conditions; and
repeating steps a) and b) until a predetermined number of polymer elements have been prepared.

8. The method of claim 1, wherein the substrate comprises a material selected from the group consisting of glass, plastic, metal, ceramic, and combinations thereof.

9. The method of claim 1, wherein the step of providing a substrate surface comprises modifying a surface chemistry of the substrate.

10. The method of claim 9, wherein modifying a surface chemistry comprises a member of the group consisting of introducing crystallographic texture, oxidizing, sulfidating, patterning, covalently attaching a functional group, and any combination of the above.

11. The method of claim 9, wherein modifying a surface chemistry comprises depositing a polymer on the surface.

12. The method of claim 11, wherein the polymer is cytophobic.

13. The method of claim 11, wherein the polymer is a hydrogel.

14. The method of claim 1, wherein the polymer elements are bound to the substrate surface via an interaction selected from the group consisting of covalent interactions, chemical adsorption, hydrogen bonding, surface interpenetration, ionic bonding, van der Waals forces, hydrophobic interactions, magnetic interactions, dipole-dipole interactions, mechanical interlocking, and combinations of these.

15. The method of claim 1, wherein the polymer elements are non-biocompatible.

16. The method of claim 1, wherein the polymer elements are biocompatible.

17. The method of claim 16, wherein the polymer elements include monomers of polymers selected from polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, chlorosulphonated polyolefins, polyaniline, polyesters, polyamides, polymerized vinyl compounds, and polymerized vinylidine compounds.

18. The method of claim 1, further comprising including a compound selected from the group consisting of drugs, growth factors, combinatorial compounds, proteins, polysaccharides, polynucleotides, and lipids in at least a portion of the polymer elements.

19. The method of claim 18, wherein the compound is covalently attached to at least a portion of the polymer elements.

20. The method of claim 19, wherein the compound is functionalized with a moiety that is incorporated into the polymer element during polymerization, and wherein the step of including comprises depositing the functionalized compound on at least one predetermined discrete monomer element.

21. The method of claim 20, wherein the moiety is a member of an acrylate group, a vinyl group, an acrylamide, and an epoxide.

22. The method of claim 20, wherein the moiety includes a photoreactive chemical group that initiates polymerization upon exposure to UV light.

23. The method of claim 19, wherein the compound is incorporated into a backbone of the polymer of the polymer element.

24. The method of claim 18, wherein the compound is non-covalently bound to the polymer of the polymer element.

25. The method of claim 1, wherein the polymer elements are spaced at intervals between about 300 μm and about 1200 μm.

26. The method of claim 1, wherein the polymer elements are spaced at intervals of less than about 300 μm.

27. The method of claim 1, wherein the polymer elements are spaced at intervals of less than about 1 μm.

28. The method of claim 1, wherein the polymer elements are spaced at intervals of less than about 0.1 μm.

29. The method of claim 1, further comprising seeding cells on the polymer elements.

30. The method of claim 29, wherein the cells are selected from the group consisting of yeast cells, mammalian cells, bacterial cells, and plant cells.

31. The method of claim 30, wherein said cells are selected from the group of mammalian cells consisting of chondrocytes, fibroblasts, connective tissue cells, epithelial cells, endothelial cells, cancer cells, hepatocytes, islet cells, smooth muscle cells, skeletal muscle cells, heart muscle cells, kidney cells, intestinal cells, organ cells, lymphocytes, blood vessel cells, stem cells, human embryonic stem cells, and mesenchymal stem cells.

32. The method of claim 1, wherein the step of depositing is performed with a robotic liquid handling device.

33. The method of claim 32, wherein the liquid handling device deposits via a member of the group consisting of pin fluid deposition, syringe pumped fluid deposition, and piezoelectric fluid deposition.

34. The method of claim 1, wherein the monomers are deposited as drops of between about 0.1 and about 100 nL.

35. The method of claim 34, wherein the monomers are deposited as drops of between 1 and 10 nL.

36. The method of claim 1, wherein the monomers are deposited as drops of less than about 0.1 nL.

37. The method of claim 1, wherein the initiating conditions are selected from the group consisting of exposure to UV light, an increase in temperature, exposure to an environment containing water vapor, exposure to an environment containing oxygen, and any combination of the above.

38. The method of claim 1, further comprising depositing a chemical initiator on the discrete monomer elements, wherein the chemical initiator is co-deposited with at least a portion of the monomers, deposited separately from at least a portion of the monomers, or co-deposited with a first portion of the monomers and deposited on the discrete elements separately from a second portion of the monomers.

39. The method of claim 38, wherein the initiator is selected from a radical initiator, a redox initiator, a thermal initiator, and an ionic initiator.

40. The method of claim 1, wherein the monomers are selected from the group consisting of 1,4 butanediol dimethacrylate, diethylene glycol diacrylate, diethylene glycol dimethacrylate, 1,6 hexanediol diacrylate, neopentyl glycol diacrylate, phenylene diacrylate 1,3, propoxylated neopentyl glycol diacrylate, tetraethylene glycol diacrylate, tetraethylene glycol dimethacrylate, triethylene glycol diacrylate, triethylene glycol dimethacrylate, tripropylene glycol diacrylate, caprolactone 2-(methacryloyloxy)ethyl ester, 5-ethyl-5-(hydroxymethyl)-β,β-dimethyl-1,3-dioxane-2-ethanol diacrylate, 1,6-hexanediol propoxylate diacrylate, 3-hydroxy-2,2-dimethylpropyl 3-hydroxy-2,2-dimethylpropionate diacrylate, glycerol 1,3-diglycerolate diacrylate, glycerol dimethacrylate, mixture of isomers, tech., 85%, neopentyl glycol dimethacrylate, neopentyl glycol ethoxylate (1 EO/OH) diacrylate, trimethylolpropane benzoate diacrylate, 1,14-tetradecanediol dimethacrylate, tricyclo[5.2.1.0^{2,6}]decanedimethanol diacrylate, trimethylolpropane ethoxylate (1 EO/OH) methyl ether diacrylate, and trimethylolpropane triacrylate, tech.

41. A microarray of polymers comprising a plurality of discrete polymer elements bound to a surface, said microarray being produced by the steps of:
providing solutions of monomers of polymer materials in a liquid phase;
depositing at least a first portion of said monomers as a plurality of discrete monomer elements on said surface; and
depositing at least a second portion of said monomers on a portion of said discrete monomer elements; and
exposing the plurality of discrete monomer elements to initiating conditions so that polymer elements are formed.

42. The microarray of claim 41, wherein a portion of said solutions include a biomolecule derivatized with a moiety that is covalently incorporated into the polymer element after the step of exposing.

43. The microarray of claim 42, wherein the moiety is selected from the group consisting of an acrylate group, a vinyl group, an acrylamide, and an epoxide.

44. The microarray of claim 42, wherein the moiety includes a photoreactive chemical structure that initiates polymerization after the step of exposing.

45. The microarray of claim 41, wherein at least a portion of the monomers are a liquid at room temperature.

46. The microarray of claim 45, wherein the liquid is combined with a solvent.

47. The microarray of claim 41, wherein at least a portion of the monomers are a solid at room temperature and providing the monomers a liquid phase comprises dissolving the monomers in a solvent at a concentration of about 3M or less.

48. The microarray of claim 41, wherein the plurality of discrete polymer elements are a first portion of the microarray of polymer elements, and wherein the steps of depositing and exposing are repeated to prepare a second portion of the array of polymer elements.

49. The microarray of claim 41, wherein each discrete monomer element is deposited and exposed individually.

50. The microarray of claim 41, wherein the surface comprises a material selected from the group consisting of glass, polymer, metal, ceramic, and combinations thereof.

51. The microarray of claim 50, wherein the surface is cytophobic.

52. The microarray of claim 50, wherein the surface is a hydrogel.

53. The microarray of claim 41, wherein a chemistry of the surface is modified.

54. The microarray of claim 53, wherein the chemistry of the surface is modified by a member of the group consisting of introducing crystallographic texture, oxidizing, sulfidating, patterning, covalently attaching a functional group, and any combination of the above.

55. The microarray of claim 41, wherein the polymer elements are bound to the surface via an interaction selected from the group consisting of covalent interactions, chemical adsorption, hydrogen bonding, surface interpenetration, ionic bonding, van der Waals forces, hydrophobic interactions, magnetic interactions, dipole-dipole interactions, mechanical interlocking, and combinations of these.

56. The microarray of claim 41, wherein the polymer elements are non-biocompatible.

57. The microarray of claim 41, wherein the polymer elements are biocompatible.

58. The microarray of claim 57, wherein the polymer elements include monomers of polymers selected from polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, chlorosulphonated polyolefins, polyaniline, polyesters, polyamides, polymerized vinyl compounds, and polymerized vinylidine compounds.

59. The microarray of claim 41, further comprising including a compound selected from the group consisting of drugs, growth factors, combinatorial compounds, proteins, polysaccharides, polynucleotides, and lipids in at least a portion of the polymer elements.

60. The microarray of claim 59, wherein the compound is covalently attached to at least a portion of the polymer elements.

61. The microarray of claim 60, wherein the compound is functionalized with a moiety that is incorporated into the polymer element during polymerization, and wherein the functionalized compound is deposited on at least one predetermined discrete monomer element.

62. The microarray of claim 61, wherein the moiety is a member of an acrylate group, a vinyl group, an acrylamide, and an epoxide.

63. The microarray of claim 61, wherein the moiety includes a photoreactive chemical group that initiates polymerization upon exposure to UV light.

64. The microarray of claim 60, wherein the compound is incorporated into a backbone of the polymer of the polymer element.

65. The microarray of claim 59, wherein the compound is non-covalently bound to the polymer of the polymer element.

66. The microarray of claim 41, wherein the polymer elements are spaced at intervals between about 300 μm and about 1200 μm.

67. The microarray of claim 41, wherein the polymer elements are spaced at intervals of less than about 300 μm.

68. The microarray of claim 41, wherein the polymer elements are spaced at intervals of less than about 1 μm.

69. The microarray of claim 41, wherein the polymer elements are spaced at intervals of less than about 0.1 μm.

70. The microarray of claim 41, wherein cells are seeded on the polymer elements.

71. The microarray of claim 70, wherein the cells are selected from the group consisting of yeast cells, mammalian cells, bacterial cells, and plant cells.

72. The microarray of claim 71, wherein said cells are selected from the group of mammalian cells consisting of chondrocytes, fibroblasts, connective tissue cells, epithelial cells, endothelial cells, cancer cells, hepatocytes, islet cells, smooth muscle cells, skeletal muscle cells, heart muscle cells, kidney cells, intestinal cells, organ cells, lymphocytes, blood vessel cells, stem cells, human embryonic stem cells, and mesenchymal stem cells.

73. The microarray of claim 41, wherein the steps of depositing are performed with a robotic liquid handling device.

74. The microarray of claim 73, wherein the robotic liquid handling device deposits via a member of the group consisting of pin fluid deposition, syringe pumped fluid deposition, and piezoelectric fluid deposition.

75. The microarray of claim 41, wherein the monomers are deposited as drops of between about 0.1 and about 100 nL.

76. The microarray of claim 75, wherein the monomers are deposited as drops of between 1 and 10 nL.

77. The microarray of claim 41, wherein the monomers are deposited as drops of less than about 0.1 nL.

78. The microarray of claim 41, wherein the initiating conditions are selected from the group consisting of exposure to UV light, an increase in temperature, exposure to an environment containing water vapor, exposure to an environment containing oxygen, and any combination of the above.

79. The microarray of claim 41, wherein a chemical initiator is deposited on the discrete monomer elements, wherein the chemical initiator is co-deposited with at least a portion of the monomers, deposited separately from at least a portion of the monomers, or co-deposited with the first portion of the monomers and deposited on the discrete elements separately from the second portion of the monomers.

80. The microarray of claim 79, wherein the initiator is selected from a radical initiator, a redox initiator, a thermal initiator, and an ionic initiator.

81. The microarray of claim 41, wherein the monomers are selected from the group consisting of 1,4 butanediol dimethacrylate, diethylene glycol diacrylate, diethylene glycol dimethacrylate, 1,6 hexanediol diacrylate, neopentyl glycol diacrylate, phenylene diacrylate 1,3, propoxylated neopentyl glycol diacrylate, tetraethylene glycol diacrylate, tetraethylene glycol dimethacrylate, triethylene glycol diacrylate, triethylene glycol dimethacrylate, tripropylene glycol diacrylate, caprolactone 2-(methacryloyloxy)ethyl ester, 5-ethyl-5-(hydroxymethyl)-β,β-dimethyl-1,3-dioxane-2-ethanol diacrylate, 1,6-hexanediol propoxylate diacrylate, 3-hydroxy-2,2-dimethylpropyl 3-hydroxy-2,2-dimethylpropionate diacrylate, glycerol 1,3-diglycerolate diacrylate, glycerol dimethacrylate, mixture of isomers, tech., 85%, neopentyl glycol dimethacrylate, neopentyl glycol ethoxylate (1 EO/OH) diacrylate, trimethylolpropane benzoate diacrylate, 1,14-tetradecanediol dimethacrylate, tricyclo[5.2.1.0^{2,6}]decanedimethanol diacrylate, trimethylolpropane ethoxylate (1 EO/OH) methyl ether diacrylate, and trimethylolpropane triacrylate, tech.
